(19) United States
(12) Patent Application Publication
(10) Pub. No.: US 2008/0045882 A1
(43) Pub. Date: Feb. 21, 2008

(54) Biological Cell Acoustic Enhancement and Stimulation

(76) Inventor: P. Michael Finsterwald, Phoenix, AZ (US)

Correspondence Address: THE LAW OFFICE OF JOHN A. GRIECCI, 703 PIER AVE., SUITE B #657, HERMOSA BEACH, CA, 90254, US

(21) Appl. No.: 11/661,340

(22) PCT Filed: Aug. 26, 2005
(86) PCT No.: PCT/US05/30464

§ 371 Date: Feb. 26, 2007

Related U.S. Application Data
(60) Provisional application No. 60/604,913, filed on Aug. 26, 2004.
(60) Provisional application No. 60/612,017, filed on Sep. 22, 2004.
(60) Provisional application No. 60/681,387, filed on May 16, 2005.

(51) Int. Cl. A61N 7/00

(52) U.S. Cl. 604/22

(57) ABSTRACT

A method for enhancing the uptake of a therapeutic biological agent by treated cells. A low-power, unfocused field of acoustic energy is directed at the treated cells after the delivery of the therapeutic agent to the treated cells. A related method for stimulating either neural cells or cells in a cell culture. A portable sized device provides the field, and may include either an array of emitters or a scanable emitter.

This application claims the benefit of U.S. provisional Application No. 60/604,913, filed Aug. 26, 2004,of U.S. provisional Application No. 60/612,017, filed Sep. 22, 2004, and of U.S. provisional Application No. 60/681,387, filed May 16, 2005. Each of the aforementioned applications are incorporated herein by reference for all purposes.

The present invention relates generally to devices and methods for the use of sonic energy to stimulate cells, and, more particularly, to devices and methods for the acoustic enhancement of biological therapies and stimulation of biological cell function.

### BACKGROUND OF THE INVENTION

In the medical arts, the efficacy of various biological therapies is often dependent upon the ability of the patient's cells to interact with the biological media used in the therapy. Moreover, both research and implementation of biological therapies may be limited by the availability of the biological media. The stimulation of cells, whether to improve their interaction with biological media, to improve their development, or even to improve their interaction with their surroundings in their normal biological location, would therefore be advantageous if it can be done in a safe and cost-effective manner.

More particularly, the rapidly developing field of cell and viral therapy is making significant progress in the treatment of illness and injury. Biological therapies are being used and are being rapidly developed for a wide range of treatments for cancer of all types, Alzheimer's, Parkinson's, and Lou Gehrig's disease, cerebral palsy, diabetes, and many other human diseases and disorders. These biological therapies include cellular therapies in which cells which have been extracted from the human or animal body and are modified, combined with other cells or agents, concentrated, and or from which other cells are derived and delivered to a patient for treatment of a medical illness or injury. An example of a rapidly developing therapy is in the use of human embryonic or adult stem cells that can repair or develop into human tissue and organs. Biological therapies also include viral therapies in which a specially engineered virus is used attack specific cells, for example cancer cells in the body. In these biological therapies it is desirable to accelerate the metabolic processes and to promote the interaction of the cells or virus with the targeted area of the body under treatment.

The efficiency of any biological therapy is important and it is desirable to reduce the treatment time and improve the effectiveness of the treatment. There is a need to improve the efficiency of biological therapies to for example to reduce the number of cells used in treatment, reduce the concentration of oncolytic virus for example, to minimize damage to surrounding tissue, and to maximize the effectiveness of treatment in a specific targeted area of the body.

The healthy function of the human body is dependent on the ability of neurons within the brain to conduct electrochemical processes that regulate and control activity within the body. In neural disorders such as epilepsy and depression the electrical activity within the brain is interrupted or becomes unregulated or uncontrolled. In neural diseases such as Alzheimer's or Parkinson's disease neurons die or become sufficiently damaged so as to no longer be able to conduct the electrochemical processes required for memory, motor function and other normal brain function.

Neurons are excitable cells in that they are stimulated to produce a small electric current. Electric current is generated by the flow of sodium and potassium ions across the cell membrane. External stimuli such as mechanical stretching and the presence of neurotransmitters further control the ionic exchange and flow of electric current between neurons and surrounding cells.

In normal brain function the capacity of neurons to transmit electrical energy is controlled by the body's ability to maintain the chemical balance of ions across the cell membranes and the maintenance of neurotransmitters such as serotonin, histamine and acetylcholine. In neural disorders and neural disease the body loses this ability to maintain chemical balance either by hyperactivity of in regions in the brain or by loss of activity through chemical imbalance or loss of cell function.

The most common form of treatment for neural disorder is drug therapy in which drugs are used to supplement or make up for the body's inability to produce certain chemical compounds or to act on various inhibitors that reduce normal brain function. Electrical stimulation is also used to externally control electrical activity in the brain. Negative side effects may result from drug therapies and surgery is often required for electrical stimulation. There is a need for an improved therapy that reduces the level of drugs required for or eliminates the need for supplemental drugs or external electrical stimulation. There is also a need for a therapy that enhances the body's own ability to maintain chemical balance and production of neurotransmitters.

A challenging problem in using cells, for example stem cells, to treat medical conditions is that it is difficult to grow sufficient number of cells in a laboratory. Donor numbers are limited and it is desirable to improve the ability to reproduce or expand the number of cells available. A common problem is that as cells multiply and form in colonies, the growth of desirable embryonic cell types is halted and the cells begin to transform or differentiate into other types of cells which are not desired for use in cell treatments. Cells such as stem cells are grown by placing a number of starter cells into a growth media and incubating the cells. As the cells grow they are later divided and reduced in concentration and new growth media is added or replaced. Cells are separated so that only the desired cell types are kept in culture.

As suggested above, the stimulation of cells, to address these needs, would therefore be advantageous if they can be done in a safe and cost-effective manner.

Sound is widely used in medicine. Focused ultrasound, i.e., a focused beam of acoustic energy in the frequency range above the range of human hearing (>20 kHz), is commonly used in medical diagnosis and treatment. Ultrasound has been widely used in medicine to image soft tissue due the high- resolution capability of focused ultrasound and the relatively low attenuation of sound the body for commonly used frequencies. Ultrasonic imaging utilizes low power levels <1 W/cm2. Frequencies of 2.5-10 MHz are generally used for diagnostic medical imaging for abdominal, cardiac, and ophthalmic imaging.

Low- frequency (1-2 MHz) ultrasound has been shown to be effective in enhancing wound healing and bone growth. High-power ultrasound or lithotripsy is commonly used for treatment of kidney stones to ultrasonically pulverize the kidney stones so the smaller particles may then pass through the patients system. The use of low frequency ultrasound in physical therapy and sports medicine is common treatment for the relief of muscle pain and to promote healing of damaged tissue. Acoustic energy has been used for thermal radiation therapy. Ultrasound some application has also been used for lipolysis of fat, as is described in the European patent application EP1060728 A1, published on Dec. 20, 2000, which is incorporated herein by reference for all purposes.

Accordingly, there has existed a need for improved devices and methods for treating cells and/or enhancing biological therapies. Preferred embodiments of the present invention satisfy these and other needs, and provide further related advantages.

### SUMMARY OF THE INVENTION

In various embodiments, the present invention solves some or all of the needs mentioned above, providing devices and methods for treating cells and/or enhancing biological therapies.

The method of the invention pertains to enhancing the uptake of a therapeutic biological agent by treated cells. A low-power, unfocused field of acoustic energy is directed at the treated cells after the delivery of the therapeutic agent to the treated cells. The invention also features a related method for stimulating either neural cells or cells in a cell culture. The invention further features a portable sized device configured to provide the acoustic field, and may include either an array of emitters or a scanable emitter.

Typically, embodiments of this invention combine the application of acoustic energy with a biological therapy, such as cell, gene, or viral therapy, to enhance the effectiveness of the biological treatment and to accelerate patient recovery. Ultrasonic waves at low power and low frequency are transmitted into the body and specifically targeted at cells or for example a gene mediated virus implanted in the body for treatment of injury, disease or illness. By transmitting sound waves into the specific area of the body; tissue, organ, blood, or bone, in which cells or virus have been transplanted, metabolism is increased and the interaction of the cells or virus with the surrounding tissue is accelerated and made more efficient. An embodiment of the present invention uses acoustic energy or sound waves to stimulate neurons surrounding cells within the brain to improve their ability to function normally. Under the invention is a method and system for applying low power, uniform field acoustic energy to a site of biological treatment in the body after implantation or delivery of the biological agent to the body to promote uptake by the surrounding tissue or cells.

Another feature of the invention is to apply ultrasound to cells such as stem cells in culture to increase the yield and to control the differentiation of cells. Low power ultrasound at a specified frequency, power level, and duty cycle may impart mechanical strain on the cells in culture and enhance the mixing of cells with the culture media to increase the rate of reproduction of viable cells. Increased production yields and accelerated expansion of cells is essential to make large quantities of cells available for cell therapies.

Other features and advantages of the invention will become apparent from the following detailed description of the preferred embodiments, taken with the accompanying drawings, which illustrate, by way of example, the principles of the invention. The detailed description of particular preferred embodiments, as set out below to enable one to build and use an embodiment of the invention, are not intended to limit the enumerated claims, but rather, they are intended to serve as particular examples of the claimed invention.

### BRIEF DESCRIPTION OF THE DRAWINGS

FIG. 1 is a perspective view of an acoustic energy stimulator embodying the invention.

FIG. 2A is a partial top view of the acoustic energy stimulator depicted in FIG. 1.

FIG. 2B is a partial side view of the acoustic energy stimulator depicted in FIG. 1.

FIG. 3 is a perspective view of the acoustic energy stimulator depicted in FIG. 1, as applied to a patient's arm.

FIG. 4 is an exploded perspective drawing of a second acoustic energy stimulator embodying the invention.

FIG. 5 is a partially exploded perspective view of the acoustic energy stimulator depicted in FIG. 4.

FIG. 6 is a cross-sectional end view of the acoustic energy stimulator depicted in FIG. 4.

FIG. 7 is a perspective view of an ultrasonic incubator embodying the present invention.

FIG. 8 is a perspective view of a mechanical scanner configured to transmit acoustic energy into a culture plate.

### DETAILED DESCRIPTION OF THE PREFERRED EMBODIMENTS

The invention summarized above and defined by the enumerated claims may be better understood by referring to the following detailed description, which should be read with the accompanying drawings. This detailed description of particular preferred embodiments of the invention, set out below to enable one to build and use particular implementations of the invention, is not intended to limit the enumerated claims, but rather, it is intended to provide particular examples of them.

Typical embodiments of the present invention reside in a devices and methods for acoustically treating cells and/or acoustically enhancing biological therapies for cells.

I. In Vivo Acoustic Stimulation

a) Acoustic Enhancement of Biological Therapy

Publications have described that high intensity focused ultrasound (HIFU) may be applied in the targeted delivery of gene therapy to organs or tissue by using short pulse, high energy ultrasound to produce micro-cavitation in a targeted area of the body prior to delivery of the genes. After the exposure to short duration, HIFU, therapeutic genes are then delivered to the site and uptake of the gene is increased significantly with respect to surrounding tissue that had not been insonified.

Such therapies may enhance the delivery of genes to a specific area of the body, but they do not provide a means for continued stimulation to promote healing after implantation. Furthermore, since the instruments to deliver HIFU are generally high cost and minimally portable, such therapy does not provide for a practical means to continuously treat a patient (i.e., to further stimulate the uptake by the body), such as with low power acoustic energy for an extended period of time after gene implantation.

Advantageously, the present invention utilizes acoustic energy to stimulate cellular activity to increase the efficiency and efficacy of biological therapies. It enhances biological therapies without damage to the therapeutic agent or the surrounding tissue at the site of illness or injury. It accomplishes this by applying acoustic energy to the body for an extended period of time, as required for treatment.

The present invention provides a method of stimulating an area of the body which has received biological therapy, for example implanted genes, by continuously delivering an extremely low power, uniform beam of preferably unfocused acoustic energy to the therapy site, and thereby to promote healing. The present invention also provides a means for low cost, portable (e.g., wearable) devices to deliver acoustic energy to the targeted site.

In a first embodiment of the invention, preferably unfocused ultrasonic energy is used to increase the efficiency of biological therapies. Included among the therapies are cell, gene, and viral therapies. More particularly, by transmitting acoustic waves into the body and insonifying the area of the body under treatment after transplant of cells or of a therapeutic virus, metabolism will be increased and the efficiency and success of the treatment will be improved. Examples of biological therapies within the scope of this embodiment include (but are not limited to) the transplant of islet cells in the pancreas, the transplant of stem cells (embryonic or adult) in any part of the body, virus mediated gene therapy, and the transplant of therapeutic DNA in any part of the human body.

Under this embodiment, an ultrasonic transducer or transducer array (hereinafter “the transducer”) is applied to the exterior of an area of the body that has undergone a biological therapy. The transducer applies unfocused acoustic energy toward the specific target of the therapy. Preferably, the acoustic energy is applied continuously over an extended period of time.

When transmitting sound into the body power levels must be maintained within safe limits. Power levels that may result in cavitation, hemolysis, or thermal shock must be avoided. In this embodiment, continuous wave (CW) ultrasound is used at a frequency of 500 KHz+/150 KHz and at a SPTA (spatial peak temporal average) intensity between 5 and 20 mW/cm². In alternative embodiments, other frequencies operating could be used, and/or the acoustic energy could be applied in a pulsed mode. The acoustic energy is preferably applied continuously over a treatment period from 30 minutes to 4 hours per day for a period from 1 day to 14 days.

The preferred embodiment of this invention utilizes power levels that are well within the defined safety limits for sound in the human body. More particularly, the application of acoustic energy in a treated body is maintained well within the safety limit defined by the FDA, which is 720 mW/cm² (SPTA).

A center frequency of 500 kHz is desirable due to the low absorption of acoustic energy by body tissue as heat. It is an advantage that the present invention delivers acoustic energy to the body with minimal heat generation, while utilizing pressure waves and the accompanied micro-streaming of intercellular fluid to stimulate activity between cells and therapeutic agents.

To obtain a uniform exposure of the targeted area of the body, it is desirable to eliminate or minimize the effect of standing waves in the acoustic energy. Such standing waves could result in localized areas of non-uniform intensity, such as acoustic hot-spots and dead-zones.

To this end, the waveform of the transmitted acoustic wave is sinusoidal and is swept in frequency over a range of, for example, between 400 and 600 KHz. This sweeping minimizes the possibility of standing waves in locations where the acoustic waves encounter strong reflectors, such as a bone/tissue interface, and are reflected between the transducer face and the interface. Alternatively or additionally, directed acoustic energy could be swept across the area to be treated, or it could be alternately applied from different locations.

Among the types of therapeutic biological agents within the scope of the invention are transplanted cell therapies including transplanted embryonic or adult stem cell therapies, gene therapies, drug therapies, therapeutic DNA therapies, protein therapies, enzyme therapies, and implanted neural cell therapies. Typically, the agents in these therapies will be stimulated with a field of acoustic energy is characterized by a temporal average acoustic power less than 20 mW/cm², and over a cumulative treatment time of greater than 1 hour.

b) Acoustic Stimulation of Neurological Cell Function

Publications have described that high intensity focused ultrasound (HIFU) may be used for thermal radiation therapy of the prostate gland and uterine fibroids. However, acoustic waves can also be used to stimulate the activity of cells within the body without causing significant heating. For example, acoustic pressure waves are able to enhance the migration of cells through a combination of mechanical energy transmitted to the cell wall and through thermal energy resulting from the partial absorption of the acoustic wave by the cells and surrounding tissue. Increased cellular interaction with surrounding cells and intercellular fluids acts to accelerate biological process in the body.

Advantageously, under this invention, acoustic energy is applied to a patient's brain to stimulate the activity of neural cells and to restore neural function. Acoustic energy may also be used in combination with drug, gene, and cell therapies to enhance their interaction with the brain and improve their effectiveness in restoring neural function, as was described above.

In a second embodiment of the invention, acoustic energy is used to stimulate neurological cell function. The acoustic energy is transmitted through the skull using an acoustic transducer coupled to the skin. Ultrasonic frequencies of less than 2 MHz frequency are readily transmitted through the skull with low loss. In the preferred embodiment of the present invention low frequency ultrasound energy is generated at 500 kHz using a piezoelectric transducer. However, frequencies are not limited to this range and other frequencies, for example, between 20 kHz and 5 MHz may be used.

Acoustic power of substantially less than 1000 mW/cm2 SPTA is preferably transmitted into the brain. For example, acoustic power level of 10 mW/cm2 at 400 kHz may be applied for 2 hours/day. Alternatively, 5 mW/cm2 may be applied for 4 hours/day, or a maximum of 20 mW/cm2 is applied for 1 hour/day to provide effective treatment.

The present invention is not limited to the above means for the delivery of ultrasound energy into the body as it will be appreciated by those skilled in the art that there are other methods including the use of phased array transducers which may be used to provide a controlled acoustic beam or scanned acoustic beam to a specific region of the body for treatment. Scanning the acoustic beam either mechanically or electronically into a region of the body may improve the effectiveness of the treatment by minimizing standing waves and providing uniform delivery of acoustic energy to the treatment site without damage to healthy tissue or cells.

Through the application of low frequency, low power ultrasonic energy, neurons are stimulated to polarize and conduct electricity. Ion exchange is assisted at the cell membrane due to the micro mechanical vibration from the acoustic pressure wave. The interaction of cells and neurotransmitters is promoted by the introduction of micro-streaming from the acoustic waves.

As with the first embodiment, to obtain a uniform exposure of the targeted area of the body, the waveform of the transmitted acoustic wave may be sinusoidal, and swept in frequency over a range of, for example, between 400 and 600 KHz. Alternatively or additionally, directed acoustic energy could be swept across the area to be treated, or it could be alternately applied from different locations.

c) Acoustic Enhancement of Neurological Therapy

Drug or gene therapies targeting neurons and neurotransmitters may also be enhanced by the addition of acoustic energy during delivery. Cell therapies in which neural cells or stem cells are transplanted in the brain may also benefit from the addition of acoustic energy which further promotes cell interaction and uptake of the cells in the body. The specific treatment times, frequencies, and acoustic power levels for effective treatment each of these biological therapies will depend on the nature of the therapy.

II. Devices for In Vivo Biological Therapies

Existing transducer arrangements for applying acoustic energy to the body are typically high power devices that direct focused beams of acoustic energy. Many of these devices are large and expensive, and thus have drawbacks for providing therapies to home-based patients.

Preferred embodiments of this invention are configured for conducting stimulation as described above, and are preferably highly efficient, lightweight, low-powered devices that may be battery powered so as to provide a portable delivery system that can be worn by a patient during therapy. The transducer is designed to be easily attached to the body over a target therapy region, such as by using an elastic belt, adhesive, Velcro, or other known means for maintaining a device on a patient's body. Similarly the drive electronics and re-chargeable battery are enclosed in a hand-held enclosure that may be worn by the patient, or attached to a hospital bed or equipment stand as required. These embodiments provide both safety and ease of use for patient.

a) Conformal Array Acoustic Stimulator

A third embodiment of the invention resides in an acoustic stimulator in the form of a transducer array (“the transducer”), and methods for using the acoustic stimulator. The acoustic stimulator is preferably configured to produce a uniform, unfocused, ultrasonic, acoustic field within a targeted therapy region of a patient's body. The transducer is also be designed to be coupled to the body and stay in a therapeutically appropriate position and orientation for an extended period of time, such as by the above-described means of attaching the transducer to the body.

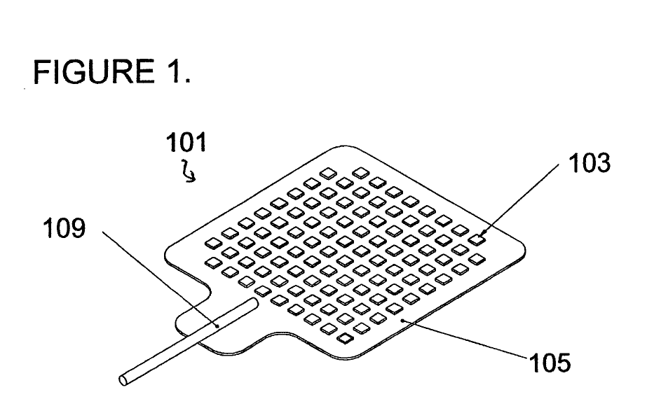

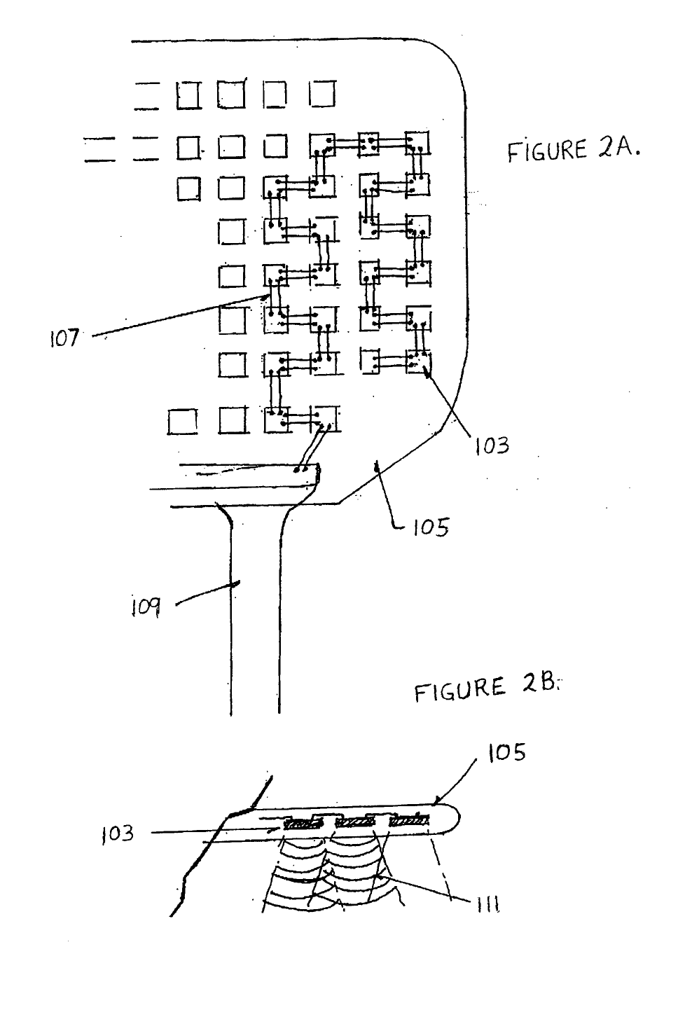

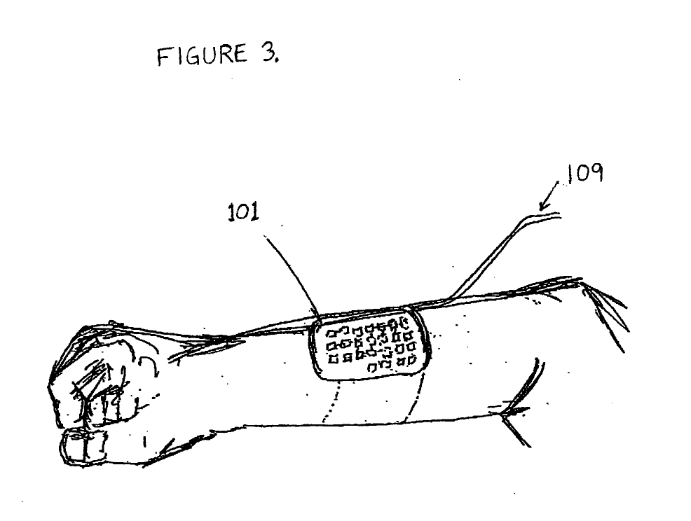

With reference to FIGS. 1-3, an acoustic stimulator 101 is in the form of a two-dimensional array of ultrasonic emitters 103 encapsulated in a thin flexible polymeric membrane 105, such as a silicone rubber membrane that is preferably (approximately) 1 mm in thickness. The emitters are separated from each other so that the array is flexible after encapsulation in the membrane. For example, the embodiment may employ individual emitters configured to be 6 mm×6 mm in size, and separated by a distance of 1 mm. Advantageously, this flexible transducer array may be able to conform over a large area of the body, which may be particularly useful in treating organs with unfocused acoustic energy.

The ultrasonic emitters 103 are micro-machined capacitive membrane ultrasound transducers (CMUT), such as those developed by Dr. Khuri-Yakub at the Ginzton Laboratory, Stanford University. An advantage of using such a device is that the thickness can be kept to a minimum and the weight is negligible compared to conventional lead- zirconate-titanate (PZT) transducers operating in the 500 KHz range.

For such a flexible transducer, the likelihood of standing waves may be less than predictable, as the array may be flexibly formed over a large area of the body with a varying shape. In order to avoid resultant hot-spots and dead-zones, the transducer is configured to alternately apply acoustic energy from different emitters 103 at different locations on the transducer. To this end, the CMUT preferably contain integrated control circuitry which allows individual emitters to turn on when a control signal is within a specified voltage or frequency range. The array is configured such that adjacent groups of emitters in the array have different control thresholds. In this way, using a single control signal, the individual emitters in the array can be driven to produce an acoustic beam 111 at different times from their adjacent emitters in the array. Thus, the constructive or destructive interference that could possibly result if all the elements were driven at one time is minimized. Optionally, groups of emitters, being located in different portions of the array, may be driven at different times. An advantage of controlling the emitters of the array in this way is that a minimum number of electrical connections 107 are necessary since all the elements in the array can more efficiently share a common control line.

In use, the flexible membrane array is conformingly placed on or over the targeted therapy region of the body, for example the abdomen. The flexible CMUT array is coupled to the body using an acoustic coupling gel, and attached using one of the aforementioned methods, such as taping the transducer to the body, or holding the transducer in place by a belt or strap around the body.

A battery and the transmit electronics necessary to control the array and drive the individual emitters (hereinafter referred to as the “control module”) are housed in a hand-held enclosure (not shown) similar in size to a portable radio, and connected to the transducer array via a miniature cable 109. The control module may also contain a timer, and may be configured to control the duration and intensity of the treatment. The enclosure can be worn by the patient on an armband, attached to the waist, or hand held, or can be attached to a hospital bed as required. A rechargeable lithium-polymer battery or equivalent is used as the battery.

b) Scanning Acoustic Stimulator

A fourth embodiment of the invention resides in an acoustic stimulator in the form of an ultrasonic transducer, and methods for using the acoustic stimulator. As was the case with the third embodiment, this embodiment is preferably configured to produce a uniform, unfocused, ultrasonic, acoustic field within a targeted therapy region of a patient's body, and is designed to be coupled to the body and stay in a therapeutically appropriate position and orientation for an extended period of time, such as by the previously-described means of attaching a transducer to a body. However, in this embodiment a single, preferably 500 KHz, transducer is mechanically oscillated about an axis of rotation 223 through a water (or other low impedance liquid) standoff 225 to sweep the acoustic energy beam through an arc.

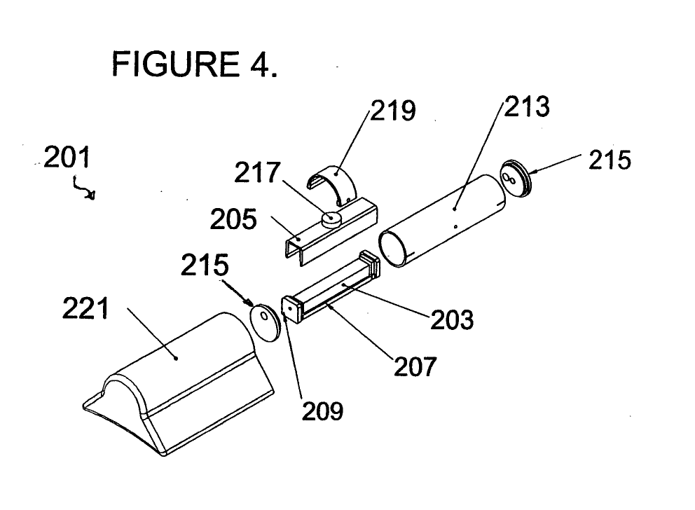

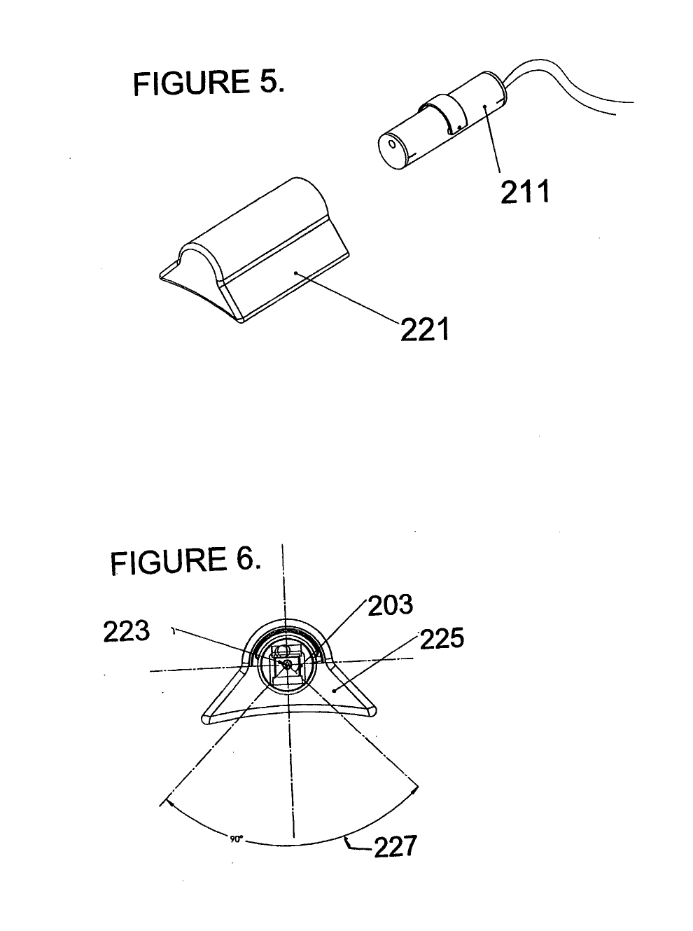

With reference to FIGS. 4-6, acoustic energy is generated by a single piezoelectric element 203 in a transducer housing 205, using a rectangular aperture of approximately 1.5 by 0.4 in. This transducer assembly is air-backed and includes a single quarter-wave matching layer 207 of copper-graphite. The transducer housing, piezoelectric element and matching layer are mounted on a shaft 209, forming a transducer assembly 211 that is supported by miniature bearings at each end of the shaft.

Electrical contact to the piezoelectric element is made by means of hairsprings attached to each end of the shaft. The hairsprings provide electrical connection to the transducer, and also serve to provide spring resistance for oscillating the transducer assembly. The transducer assembly and hairsprings are enclosed in a thin cylindrical tube made of polystyrene. The ends of the tube are sealed by end-caps that house the miniature bearings. The cylindrical tube is filled with mineral oil to provide acoustic coupling from the transducer element into the plastic tube.

Within the cylindrical tube 213, a permanent magnet 217 is mounted on the transducer housing 205, preferably halfway along the length of the transducer assembly 211. An electromagnet 219 is mounted on the outside of the cylindrical tube, and located around the portion of the cylindrical tube that contains the permanent magnet. The electromagnet may be driven sinusoidally with a current sufficient to produce an alternating magnetic field that can act on the permanent magnet to drive the transducer assembly to oscillate about an axis extending between the miniature bearings, at a resonant frequency of the transducer assembly/hairsprings system.

Preferably, the resonant frequency of the oscillating transducer assembly working against the hairsprings is designed to be in the range of 1 to 5 Hz. The electromagnet 219 is driven so as to oscillate the transducer assembly 211 through a scan angle 2 27 of ±45 degrees to provide a total scan angle of 90 degrees. In this manner a wide field of transmission (90 degrees) can be delivered through a relatively small area of contact with the body. The complete assembly of the transducerassembly, cylindrical tube and electromagnet (hereinafter referred to as the “micro-scanner”) is encapsulated in an interface member 221, such as a silicone rubber interface, that serves to couple the acoustic energy from the cylindrical tube into the patient's skin.

In use, this acoustic stimulator 201, i.e., the micro-scanner encapsulated in the silicone rubber interface 221, is attached to apatient's body, e.g., to the center of the patient's forehead using one of the aforementioned means of attachment, such as an elastic headband. Prior to placing the acoustic stimulator on the forehead a small amount of acoustic coupling gel or lotion is applied to a body-contact surface of the interface member, or to the skin.

In order to avoid hot-spots and dead-zones, the acoustic stimulator 201 is configured to oscillatingly direct its unfocused acoustic energy back and forth over the area of interest. The ultrasound transducer is driven with a sine wave pulse burst, 5 cycles with a period of 50 msec (50% duty cycle).

The piezoelectric element and drive electronics for the electromagnet are battery powered. A rechargeable battery and the drive electronics (hereinafter referred to as the “control module”) are housed in an enclosure and connected to the micro-scanner via a miniature cable. The control module can be worn by the patient on an armband, attached to the waist, or hand held, or can be attached to a hospital bed as required. The control module may also contain a timer which may control the duration of the treatment and automatically turn off the transducer pulser and the electromagnetic drive after a pre-set amount of time.

Advantageously, the present embodiment provides acoustic energy scanned in a sector format, which provides for a more uniform delivery of acoustic energy to a patient. Also, the present embodiment requires only a small contact area on a patient's body, and therefore the acoustic beam is not adversely affected by time-of- flight variations, e.g., due to irregularities in the human skull. Thus, while the scanning acoustic stimulator described above may have wide application in the treatment of biological therapies in all parts of the body, it may be particularly useful for the application of ultrasound for the treatment of the brain.

In variations of this embodiment, the actuator for oscillation of the transducer assembly may be a dc motor, or other typical actuation devices, as are known. Optionally a position sensor and control system may be used to actively control the oscillation using a feedback control servo loop. Additionally, it is within the scope of the invention to provide the device with a second axis of rotation, thereby offering better control over the area in which acoustic energy is delivered. Alternatively, oscillation may be stimulated through the use of a phased array acoustic energy device.

III. In Vitro Cell Culture

It has previously been shown that imparting mechanical strain to stem cells, such as by stretching a flexible matrix carrying stem cells, is an effective aid to culturing the stem cells. Acoustic energy may be used to enhance the growth of cells in culture. More particularly, ultrasound can be used to impart mechanical strain on the cells and facilitate mixing and interaction of the cells with the culture media. By exposing the cells in culture to ultrasound they can be kept in an active state thus inhibiting the differentiation of cells into less desirable cell types.

In a fifth embodiment of the invention, preferably unfocused acoustic energy is used to stimulate the development of cells, such as stem cells, in a cell culture. More particularly, continuous wave ultrasound is used at a frequency of 500 KHz±/150 KHz and at a SPTA (spatial peak temporal average) intensity between 10 and 50 mW/cm² to stimulate the development of cells in a cell culture. The ultrasound is applied continuously during the incubation of the cells in a culture to stimulate the interaction of cells with the culture media, and thereby increase the metabolic activity of the cells. The invention is not limited to stem cells, as the culture of other types of biological cells may similarly be enhanced by application of acoustic energy.

Preferably, the cell culture container and acoustic energy stimulator are configured to produce a uniform acoustic field within the cell culture and minimize or eliminate standing waves, so as to obtain a uniform exposure of the cells to the acoustic energy. Therefore, in a preferred version of this embodiment, the waveform of the transmitted acoustic wave is sinusoidal and is swept in frequency over a range of for example between 400 and 600 KHz. This method of insonification will generally minimize standing waves in situations where the acoustic waves encounter strong reflectors, such as at the liquid/air interface that occurs at the wall of a culture dish. Preferably, the cell culture is stimulated by a stimulator driven at 500 KHz in a pulse mode, delivering 100 pulses of 1 W/cm² each at a pulse repetition of 1 millisecond yielding a 20% duty cycle and SPTA=20 mW/cm².

IV. Devices for In Vitro Cell Culture

a) Cell Culture Acoustic Energy Stimulator

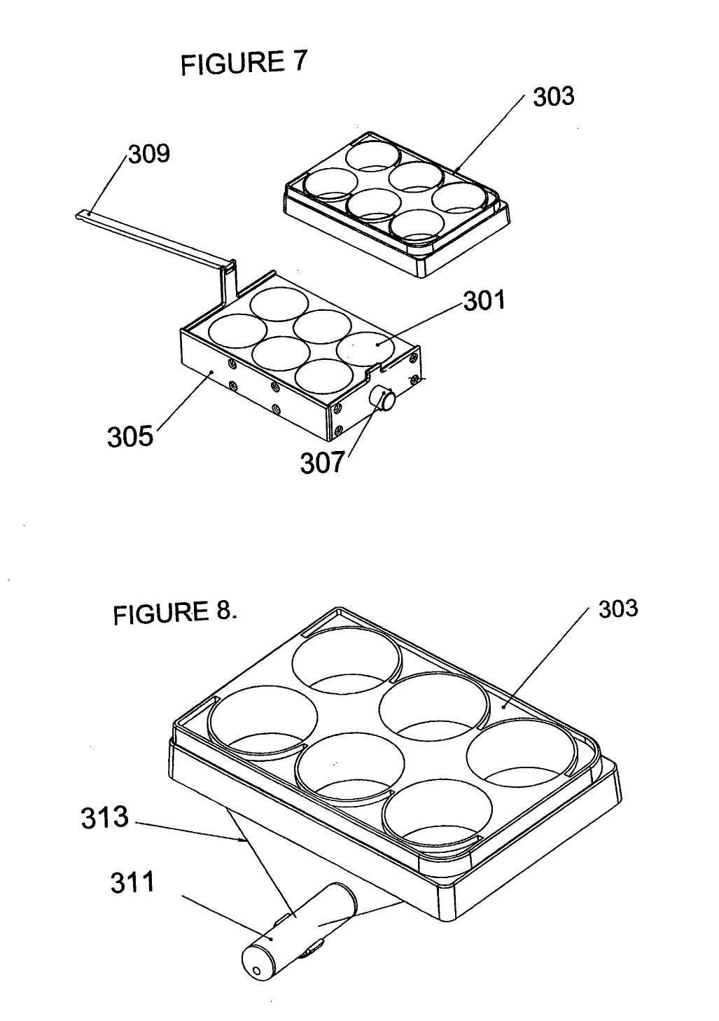

In light of the fifth embodiment, the scope of the invention includes a means for applying sound to cells in culture. With reference to FIG. 7, in a sixth embodiment of the invention, a 500 KHz center frequency single element PZT transducer 301 is used with a single quarter wave matching layer made of copper impregnated graphite to stimulate a cell culture. The transducer is air-backed to provide maximum efficiency. The front layer of the transducer is a silicone rubber sheet 1/16 in. thickness. The transducer is coupled to the bottom side of a standard 6 well culture plate 303, such as that manufactured by COSTAR®. Six individual transducers are mounted in a base plate 305 that locates the transducers to align with each of the six wells in the culture plate. Acoustic coupling gel is placed on a silicone rubber surface of the each transducer to increase acoustic coupling between that surface and a bottom wall of the culture plate. A signal generator (not shown), such as HP 33220A, by AGILENT TECHNOLOGIES®, is connected to the base plate via an electrical connector 307, and is used to drive the individual transducers.

The base plate 305 is configured to accept the six well culture plate. The base plate includes features such as flanges for self locating the culture plate in proper alignment to the individual transducers. The base plate also includes a means for securing the culture plate in place during use, such as a locking arm 309 configured to hold the culture plate to the base plate.

In a variation of this embodiment, the drive electronics (not shown) and a re-chargeable battery (not shown) are located within the transducer base plate 305, allowing for the assembled culture plate and transducer base to be used without electrical wiring, a separate signal generator, or wires for attachment to the separate signal generator.

With reference to FIG. 8, in an alternative embodiment, a mechanical sector scanner 311, similar to one described above, may be used to deliver a uniform unfocused acoustic beam 313 into the various wells of the culture plate. The mechanical scanner is coupled to culture plate by a water path or coupling medium (not shown) such as silicone rubber. An advantage of this arrangement is that by scanning a uniform acoustic beam into the culture plate the cells are exposed to a more uniform sound field and thus the growth of the cells within the individual chambers are more uniform within. In the preferred embodiment of this invention the scanner scans the culture plate at a frequency of 5 Hz and delivers 10 mw/cm² SPTA acoustic power at the surface of the individual wells.

b) Culture Plate for Cell Culture Acoustic Energy Stimulator

In the seventh embodiment of the invention, a culture plate is provided with various means to improve the delivery of acoustic energy, and minimize the development of standing waves during acoustic energy delivery. The means may include features to provide features for locating and holding the culture plate to the transducer base plate, e.g., flanges, grooves, ridges and/or the like. Additionally the culture plate may be configured to optimize the transfer of acoustic energy from the transducers, for example by having an elastomeric layer, e.g., silicone rubber, as the bottom of the culture plate instead of a being of a rigid plastic construction, and/or by matching the impedance of the culture plate materials to the transducer and the target.

c) Large Scale Cell Culture Acoustic Energy Stimulator

In order to apply the current invention to the large scale expansion of cells such as stem cells, it is necessary to use larger culture vessels. To stimulate cells within such a vessel, enlarged variations of the above-discussed technology may be used. Alternatively, the cells may be maintained in a system wherein a portion of the cells are continuously pumped through an acoustic stimulation chamber.

In these embodiments, in order to uniformly expose a large quantity of cells in a large volume culture media it may be beneficial to use a single scanning transducer (as described above), and scan the volume mechanically by sweeping the transducer about an axis through a water path. Alternatively an electronically steered phased array may be used to direct the ultrasound beam throughout the culture.

Alternatively, a 500 KHz single element transducer may be mechanically oscillated about an axis of rotation through a water standoff (or other low impedance liquid) to sweep the acoustic beam through an arc. For example a rectangular transducer with dimensions 5 mm×50 mm can be oscillated about an axis and through a ±45 degree angle to insonify a 90 degree×50 mm volume. A dc motor can be used to mechanically oscillate the transducer. The frequency of oscillation, the scan angle, and the acoustic power transmitted by the transducer must be taken into account to determine the temporal average sound intensity delivered to the culture.

In these embodiments, preferably ultrasonic frequencies (>20 kHz) are used and preferably in the range 350 kHz to 650 kHz and at a SPTA (spatial peak temporal average) intensity between 10 and 50 mW/cm². The present invention is not limited to acoustic energy in this frequency range, as further developments studies may determine other frequencies that are preferable.

It is to be understood that the invention comprises apparatus and methods for designing, producing and using devices and/or methods for acoustically treating cells and/or acoustically enhancing biological therapies for cells. Alternative variations of these embodiments could comprise other types of devices and methods. In short, the above disclosed features can be combined in a wide variety of configurations within the anticipated scope of the invention.

While particular forms of the invention have been illustrated and described, it will be apparent that various modifications can be made without departing from the spirit and scope of the invention. Thus, although the invention has been described in detail with reference only to the preferred embodiments, those having ordinary skill in the art will appreciate that various modifications can be made without departing from the scope of the invention. Accordingly, the invention is not intended to be limited by the above discussion, and is defined with reference to the following claims.

I claim:

1. A method for enhancing the uptake of a therapeutic biological agent by treated cells, comprising:
transmitting a low power field of acoustic energy into the treated cells after the delivery of the therapeutic agent to the treated cells.

2. The method of claim 1, wherein:
the field of acoustic energy is characterized by a temporal average acoustic power less than 20 mW/cm²; and
the step of transmitting is conducted over a cumulative treatment time of greater than 1 hour.

3. The method of claim 1, wherein the therapeutic biological agent is a cell therapy that is transplanted into a patient's body for the treatment of disease, injury or illness.

4. The method of claim 3, wherein the therapeutic biological agent is a stem cell therapy in which embryonic or adult stem cells are introduced to a part of a patient's body for the treatment of disease, injury or illness.

5. The method of claim 1, wherein:
the therapeutic biological agent is a gene therapy in which genes or gene mediated virus are implanted in a patient's body for treatment of disease, injury or illness;
the field of acoustic energy is transmitted for an extended period of time after the implantation; and
the field of acoustic energy is transmitted by means of a patient-worn acoustic transmitter.

6. The method of claim 1, wherein the therapeutic biological agent is a drug therapy in which one or more drugs are introduced to a patient's body for the treatment of disease, injury or illness.

7. The method of claim 1, wherein the therapeutic biological agent is a DNA therapy in which a therapeutic DNA is introduced to a patient's body for the treatment of disease, injury or illness.

8. The method of claim 1, wherein this therapeutic biological agent is one or more proteins introduced into a patient's body for the treatment of disease, injury or illness.

9. The method of claim 1, wherein the therapeutic biological agent is one or more enzymes introduced into a patient's body for the treatment of disease, injury or illness.

10. The method of claim 3, wherein the therapeutic biological agent is a collection of neural cells implanted into a patient's body.

11. A method for stimulating neural cells in the treatment of neural disease or neural disorder, comprising:
transmitting a low power field of acoustic energy into the human brain.

12. The method of claim 11, wherein:
the field of acoustic energy is characterized by a temporal average acoustic power of less than 20 mW/cm²; and
the step of transmitting is conducted over a cumulative treatment time of greater than 1 hour.

13. The method of claim one, wherein the low power field of acoustic energy is applied to a patient's brain to enhance the effectiveness of a biological therapy and increase the uptake of the therapeutic biological agent in the brain.

14. The method of claim 13, wherein:
the field of acoustic energy is characterized by a temporal average acoustic power less than 20 mW/cm²; and
the step of transmitting is conducted over a cumulative treatment time of greater than 1 hour.

15. The method of claim 13, wherein the therapeutic biological agent is biological cells that are transplanted into a patient's brain for the treatment of disease, injury or illness.

16. The method of claim 13, wherein the therapeutic biological agent is a collection of neural cells.

17. The method of claim 13, wherein the therapeutic biological agent is embryonic or adult stem cells transplanted into a patient's brain for the treatment of disease, injury or illness.

18. The method of claim 13, wherein the therapeutic biological agent is genes or a gene mediated virus implanted in a patient's body for treatment of disease, injury or illness.

19. The method of claim 13, wherein the therapeutic biological agent is one or more drugs introduced to a patient's body for the treatment of neural disease or neural disorder.

20. The method of claim 13, wherein the therapeutic biological agent is a therapeutic DNA introduced to a patient's body for the treatment of neural disease or disorder.

21. The method of claim 13, wherein the therapeutic biological agent is one or more introduced proteins into a patient's body for the treatment of neural disease or disorder.

22. The method of claim 13, wherein the therapeutic biological agent is one or more enzymes introduced into a patient's body for the treatment of neural disease or disorder.

23. A device for the delivery of therapeutic acoustic energy to a body comprising:
a membrane flexible enough to conform to the body; and
a two dimensional array of acoustic emitters mounted within the membrane.

24. The device of claim 23, wherein the acoustic emitters are capacitive membrane ultrasound transducers.

25. The device of claim 23, wherein the acoustic emitters are electrically connected in parallel.

26. The device of claim 24, wherein each emitter is configured to have an integral addressable switch for selectively controlling acoustic emissions.

27. The device of claim 23, in which the emitters are configured to transmit in pseudo random order to produce a non-focused emission of acoustic energy.

28. The device of claim 23, and further comprising a carryable housing containing a control system configured for driving the acoustic emitters, or in the control system and emitters are configured to be powered by a battery housed within the housing.

29. A method of enhancing a culture of biological cells during a period of cell incubation, comprising:
insonifying the biological cells with low power acoustic energy during the period of cell incubation.

30. The method of claim 29, wherein
the field of acoustic energy is characterized by a temporal average acoustic power less than 20 mW/cm²; and
the step of insonifying is conducted over a cumulative treatment time of greater than 1 hour.

31. The method of claim 29, wherein the culture of biological cells is a culture of human adult or embryonic stem cells.

32. A device for the culture of biological cells in a multiple well culture plate, comprising:
a base plate configured to receive the multiple well culture plate;
an array of acoustic emitters mounted in the base plate such that each emitter is aligned with a corresponding well on the multiple well culture plate; and
a means for electrically driving the acoustic emitters so as to transmit acoustic energy into each well of the multiple well culture plate.

33. The device of claim 32, wherein the means for electrically driving the acoustic emitters is battery powered and located within an enclosure adjoining the base plate, such that the device is a portable, self-contained system.

34. A device for the large-scale culture of biologic al cells in a culture vessel, comprising:
an acoustic emitter is coupled to a culture vessel.

35. The device of claim 34, wherein the vessel is configured for the biological cells to be human embryonic or adult stem cells.

36. The device of claim 34, wherein the acoustic emitter is a single element transducer fixed in relation to the culture vessel.

37. The device of claim 34, wherein the acoustic emitter is a mechanically scanned acoustic emitter that oscillates about one or more axes of rotation.

38. The device of claim 34, wherein the acoustic emitter is an electronically phased array of individual elements.

39. A device for the delivery of therapeutic acoustic energy, comprising:
a body forming a fluid filled chamber;
an transducer element rotatably mounted within the fluid filled chamber;
a first magnet configured as an electromagnet; and
a second magnet, wherein one of the first and second magnets is mounted to the transducer element, and the other is mounted to the body such that the first and second magnets are configured to drive the transducer element in rotation with respect to the body;

40. The device of claim 39, and further comprising an outer-housing configured of a material to acoustically couple acoustic energy from the transducer element to an external subject.

41. The device of claim 39, and further comprising:
a position sensor configured to detect the angular rotation of the transducer element with respect to the body; and
a control system configured to control the angular position of the transducer element based on a signal from the position sensor.
